(19) United States
(12) Patent Application Publication
(10) Pub. No.: US 2015/0289892 A1
(43) Pub. Date: Oct. 15, 2015

(54) Low Force Thrombectomy Device

(71) Applicant: THE HONG KONG UNIVERSITY OF SCIENCE AND TECHNOLOGY, Hong Kong (CN)

(72) Inventors: David Chuen Chun Lam, Hong Kong (CN); Chi Hang Chon, Hong Kong (CN); John Ching Kwong Kwok, Hong Kong (CN); Matthew Ming Fai Yuen, Hong Kong (CN); Zhen Qin, Hong Kong (CN); Alexander Ka Ngai Lam, Hong Kong (CN); Ka Kit Leung, Hong Kong (CN); Wai Lun Ko, Hong Kong (CN)

(21) Appl. No.: 14/443,837

(22) PCT Filed: Nov. 20, 2013
(86) PCT No.: PCT/CN2013/001420

§ 371 Date: May 19, 2015

Related U.S. Application Data
(60) Provisional application No. 61/728,822, filed on Nov. 21, 2012.

(51) Int. Cl. A61B 17/22; A61B 18/06; A61B 18/18; A61B 17/221; A61B 18/08; A61B 18/14

(52) U.S. Cl. CPC A61B 17/22; A61B 18/08; A61B 18/14; A61B 18/1815; A61B 17/22031; A61B 17/221; A61B 18/06; A61B 2018/00083

(57) ABSTRACT

The claimed biocompatible device is designed to remove vessel occlusions such as a thrombus, blood clot, or embolus. The claimed devices maybe used to treat occlusions in the brain, in the vasculature, and in tissues and organs.

The device is in a form of a wire or wire-like structure which can capture the occlusion with minimal contact force with the vessel wall by an application of electricity, or electrical, chemical, or microwave generated heat to enable capture of the occlusion by the device. The device, compared to conventional mechanical thrombectomy devices, captures the occlusion and reduces occlusion fragmentation without large radial force and vessel wall friction that can damage the vessel wall.

### CROSS-REFERENCE TO RELATED APPLICATION

The present application claims the benefit of U.S. Provisional Application Ser. No. 61/728,822, filed Nov. 21, 2012, which is hereby incorporated by reference in its entirety including any tables, figures, or drawings.

### BACKGROUND OF THE INVENTION

Blockages in arteries and veins are often caused by a thrombus, blood clot, or embolus, and can lower the perfusion of blood. If the blood perfusion is below a certain level, cells receive insufficient amounts of oxygen. If the situation is prolonged, cells will die.

A blockage in blood vessels leading to the death of brain cells is one of the causes of stroke, which can be fatal if proper medication or treatment is delayed. Current medical treatments for stroke include thrombolysis, mechanical thrombectomy, and surgery. Thrombolysis techniques are usually only effective within the first three hours of the onset of stroke symptoms. Mechanical thrombectomy devices typically utilize devices that apply a large radial force for capturing the blockage inside the blocked vessel, which may scratch on the vessel wall and damage the vessel wall and fragment the thrombotic material during the extraction, leading to downstream occlusions. Surgery is an invasive medical treatment, which requires risk and a longer recovery time.

There is a need for vessel blockage removal that is efficient and safe, allowing removal of the entire blockage without vessel wall damage or blockage fragmentation. The present invention provides a low force thrombectomy device for the removal of most blockages that occur in blood vessels. This device can minimize the problems of thrombus fragmentation, minimize the damage to the vessel wall, often caused during the blockage removal process, and increase the removal efficiency, often providing complete removal of the blockage.

### BRIEF SUMMARY OF THE INVENTION

Unlike conventional mechanical thrombectomy devices, which utilize device that apply a large radial force to the vessel wall in order to remove a thrombus, blood clot or embolus, the device of the present invention provides a device and method that minimizes the problems of blockage fragmentation, minimizes damage to the vessel wall, and allows for complete removal of the blockage.

The device of the present invention can elevate the temperature of the blockage, which leads to the adhesion of the blocking material to at least portion of the device to capture the blockage. The elevated temperature of the blocking material can increase the rigidity or firmness of the blocking material. The firmer blocking material is less likely to fragment during the extraction process, providing more complete removal of the blockage. As results, there is less likelihood to create occlusions at distal portions of the vessel due to the blockage fragments. The present invention also obviates the need to apply large radial forces and therefore reduces the potential for vessel wall damage.

The wire or wire-like structure of the device of the present invention can be made of a biocompatible material, which can generate heat, elevate the temperature of the blockage, and capture the blockage with minimal force with the vessel wall. The wire or wire-like structure of the device can also be coated or covered with one or more layers of a material or coating that insulates the thermal or electrical energy and prevents direct contact between the wire or wire-like structure and the vessel wall, which contact could result in electric shock, or overheating of the vessel wall. This insulating material or coating can also be made of a biocompatible material. In an embodiment, the material can also be transferable from the device to the vessel wall, if desired; and in other embodiments it is not so transferable.

The methods and devices of the subject invention when used for blockage removal can include navigation within a vessel, integration of the device with the blockage, elevation of temperature to the blockage, and extraction of the blockage. The devices can be navigated and deployed to the position of the targeted blockage inside a vessel by a guide wire and a catheter.

### BRIEF DESCRIPTION OF THE DRAWINGS

In order that a more precise understanding of the above recited invention can be obtained, a more particular description of the invention briefly described above will be rendered by reference to specific embodiments thereof that are illustrated in the appended drawings. The drawings presented herein may not be drawn to scale and any reference or indication of dimensions in the drawings or the following description is specific to the embodiments disclosed. Any variations of these dimensions that will allow the subject invention to function for its intended purpose are considered to be within the scope of the subject invention. Thus, understanding that these drawings depict only typical embodiments of the invention and are not therefore to be considered as limiting in scope, the invention will be described and explained with additional specificity and detail through the use of the accompanying drawings in which:

FIG. 1 shows a schematic drawing of one embodiment of the device comprising a wire or wire-like structure of biocompatible material.

FIG. 2 shows a schematic drawing of one embodiment of the device comprising a wire or wire-like structure of biocompatible material with a biocompatible coating that can include a self-heating chemical or a combination of chemicals that can generate heat when combined.

FIG. 3 shows the schematic drawing of one embodiment of the device comprising a wire or wire-like structure of a biocompatible, electrically conducting material. The electrically conducting material can further comprise a biocompatible coating made of an electrical, thermal or electrical and thermal insulated material.

FIG. 4 shows the schematic drawing of the device being a wire or wire-like structure of a biocompatible material with a biocompatible coating that can include a self-heating chemical or a combination of chemicals that can generate heat when combined, and can further be partially coated with biocompatible insulation material in the outermost layer that provides electrical, thermal or electrical and thermal insulation.

FIG. 5 shows the schematic drawing of the device integrated into the blockage material within a vessel.

FIG. 6 illustrates an embodiment having a microwave receiving material coating on a portion of the wire.

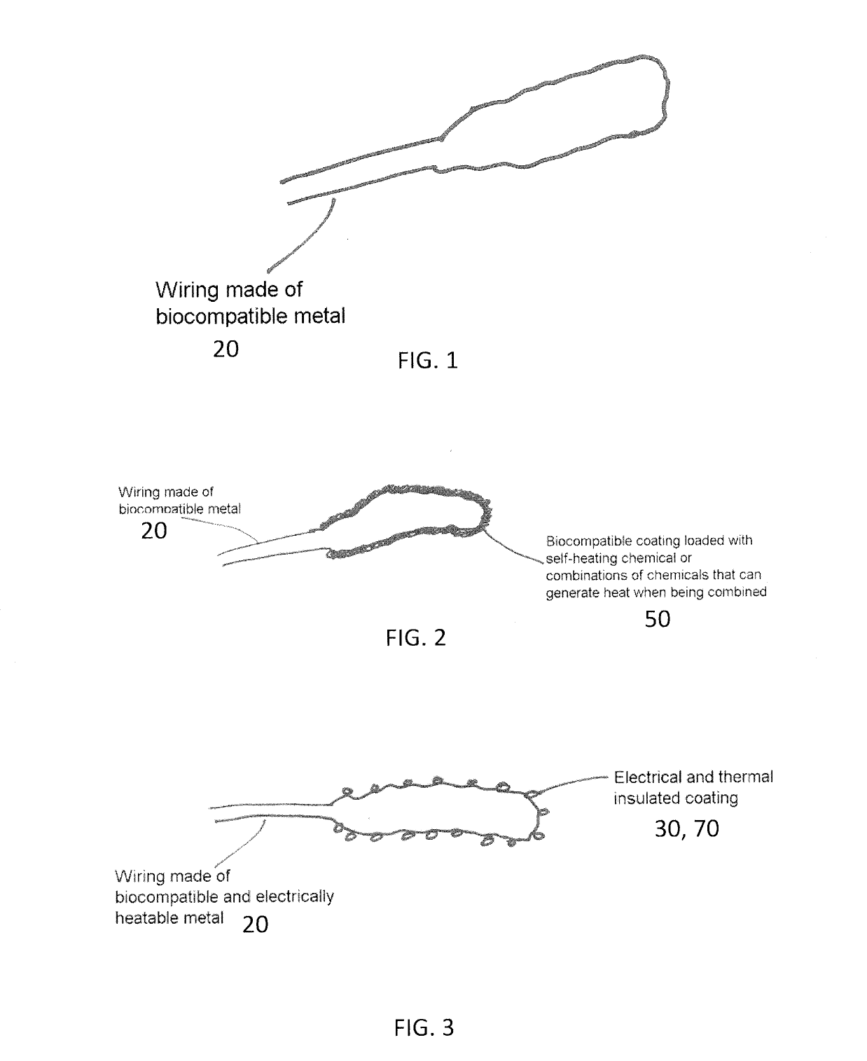

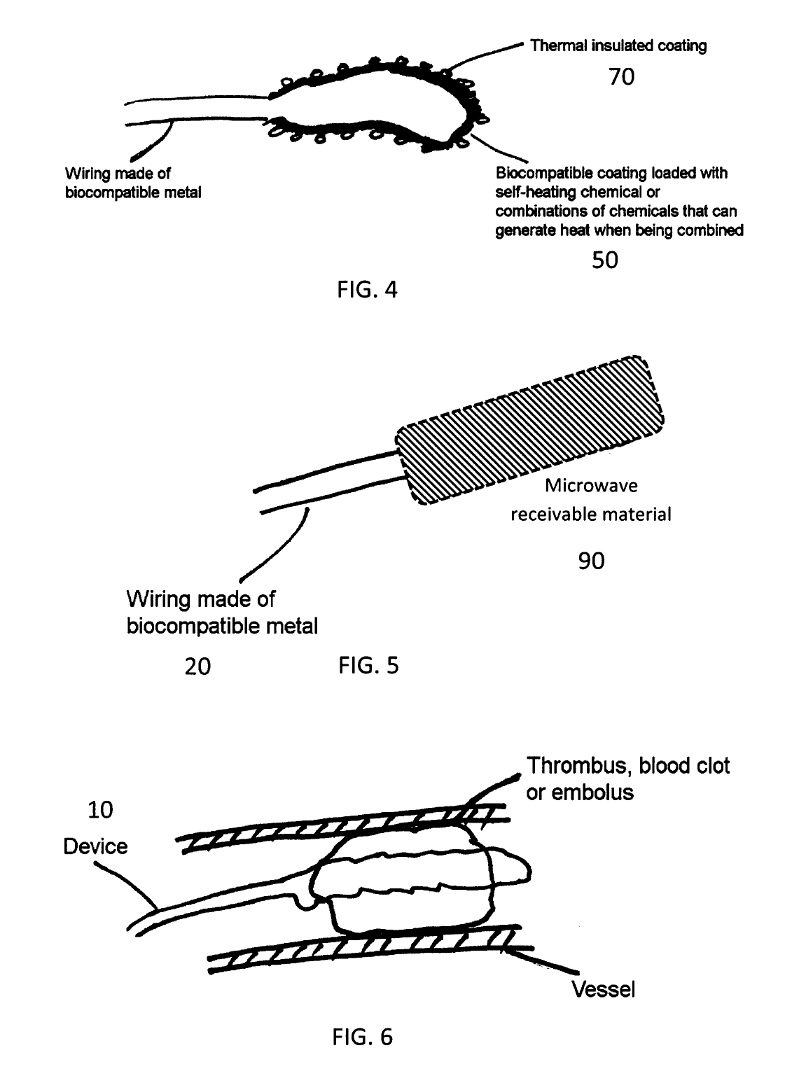

### DETAILED DISCLOSURE OF THE INVENTION

The subject invention relates to systems and methods for vessel blockage treatment. This description is not to be taken in a limiting sense, but is merely for the purpose of illustrating the general principles of the invention. Those of skill in the art will recognize that the following description is merely illustrative of the principles of the invention, which may be applied in various ways to provide many different alternative embodiments.

Current methods for treating vessel blockages include mechanical removal devices and pharmacological treatments to dissolve or break down the blockage, often combined with the use of devices or physical structures used to expand a partially open or re-opened vessel lumen within the vascular system. Common devices used to reopen partially blocked vessels include balloon angioplasty devices and stents.

The following description will disclose that the subject invention is particularly useful in the field of angiographic surgical procedures, in particular devices used for the treatment and/or removal of vessel blockage material. However, a person with skill in the art will be able to recognize numerous other uses that would be applicable to the devices and methods of the subject invention. While the subject application describes, and many of the terms herein relate to, a use for treatment of vessel blockages and/or removal of blocking material, other modifications will be apparent to a person with skill in the art having benefit of the subject disclosure and are contemplated to be within the scope of the present invention.

In the description that follows, a number of terms used in relation to angiography or blood vessels are utilized. In order to provide a clear and consistent understanding of the specification and claims, including the scope to be given such terms, the following definitions are provided.

The term “patient” as used herein, describes an animal, including mammals to which the systems and methods of the present invention are applied. Mammalian species that can benefit from the disclosed systems and methods include, but are not limited to, apes, chimpanzees, orangutans, humans, monkeys; domesticated animals (e.g., pets) such as dogs, cats, guinea pigs, hamsters; veterinary uses for large animals such as cattle, horses, goats, sheep; and any wild animal for veterinary or tracking purposes.

The present invention is more particularly described in the following examples that are intended to be illustrative only since numerous modifications and variations therein will be apparent to those skilled in the art. As used in the specification and in the claims, the singular for “a,” “an” and “the” include plural referents unless the context clearly dictates otherwise.

Reference will be made to the attached figures on which the same reference numerals are used throughout to indicate the same or similar components. With reference to the attached figures, which show certain embodiments of the subject invention, it can be seen that the subject invention comprises an angiographic device 10 for insertion into a blood vessel that includes at least a wire 20 and one or more of an electrically insulating coating 30 on the wire, one or more heat-generating chemicals 50, a thermally insulating coating 70, and/or a microwave receiving coating 90. Mechanical blockage removal devices include devices that expand around a thrombus and capture and extract the thrombotic material. These devices exert mechanical stress on the vessel wall and can cause vessel wall damage, which can further the development of atherosclerotic plaques and subsequent thrombus formation.

The drugs used in pharmaceutical blockage removal (thrombolysis) normally enter the body by intravenous therapy, which has a lower risk of vessel wall damage compared to mechanical blockage removal. However, thrombolysis requires longer treatment times and may lead to fatal side effects, such as Symptomatic Intracranial Hemorrhage. Following blockage removal, angioplasty balloons and stents may be used to preserve the diameter of an opened or recanalized vessel and prevent subsequent closure by an atherosclerotic lesion.

The present invention relates to a device for removal of vessel blockages that does not exert mechanical stress on the vessel wall, uses heat to enhance the interfacial adhesion, increase the rigidity or firmness of the blockage, and allows complete removal of the blockage material. The functional part of the present invention is made of a biocompatible wire or wire-like structure. Unlike conventional devices, in an embodiment of the subject invention, electrical current is allowed to pass through the biocompatible wire or wire-like structure. When electricity passes through the biocompatible wire-like structure, the temperature of the wire-like structure is increased. The increased temperature of the wire-like structure then elevates the temperature of the blockage. The elevated temperature of the blockage enables the wire or wire-like structure to capture the blockage with minimal force applied to the vessel wall, which minimizes the risk of vessel wall damage. The elevated temperature of the blockage also increases the rigidity or firmness of the blockage and minimizes the risk of blockage fragmentation and additional occlusions in distal portions of the vessel.

Although exemplary devices for removal of blockages in blood vessels are described, such devices can be applicable to blockages of other cavities or endoluminal ducts in a patient, which can include, but is not limited to, the cerebral circulation system, the cardiovascular system, the biliary hepatic system, the tracheobronchial system, the esophageal bowel system, and the urinary tract system. Embodiments of the invention, some of which are described herein, are readily adaptable for use in blockage removal in a variety of vessels, including, but not limited to, treatment or reopening of vascular blockages.

Stents are one of the conventional devices that are expandable prostheses employed to open and maintain vascular and endoluminal ducts or tracts that are unoccluded. For example, stents are frequently used to maintain the patency of a coronary artery after dilation by a balloon angioplasty procedure. There are a variety of stents utilized in procedures and often employ a tubular meshwork structure having an exterior surface defined by a plurality of interconnected struts and spaces between the struts. The tubular structure is generally expandable from a first position, wherein the stent can be sized for intravascular insertion, to a second position, wherein at least a portion of the exterior surface of the stent contacts and engages the vessel wall where the stent has been placed.

The expanding of the stent is usually accommodated by flexing and bending of the interconnected struts throughout the structure. The force for expansion of the stent can be applied externally as from an inflated balloon onto which the stent is loaded prior to placement, or the stent may be self-expanding. A myriad of strut patterns are known for achieving various design goals such as enhancing strength, maximizing the expansion ratio or coverage area, enhancing longitudinal flexibility or longitudinal stability upon expansion, etc. One pattern may be selected over another in an effort to optimize those parameters that are of particular importance for a particular application. While a stent may be deployed by radial expansion under outwardly directed radial pressure exerted, for example, by active inflation of a balloon of a balloon catheter on which the stent is mounted, the stent may be self-expandable. In some instances, passive spring characteristics of a preformed elastic (i.e., self-opening) stent serve the purpose, while in others shape memory materials are used, such that upon activation by the appropriate energy source, the stent deforms into a pre-determined memorized shape. Regardless of design, in all cases the stent is expanded to engage the inner lining or inwardly facing surface of the vessel wall with sufficient resilience to allow some contraction, but also with sufficient stiffness to largely resist the natural recoil of the vessel wall following deployment.

Stent deployment requires a minimal unoccluded vessel lumen. In cases of complete lumen obstruction, it is necessary to first use pharmacological treatment aimed at dissolving the blockage before introducing a balloon or stent into the wholly or partially reopened vessel lumen.

To place a stent, a guide wire lumen is used for introducing a guide wire in a balloon catheter, and a balloon inflating lumen for inflating the balloon after the stent has been placed at a desired location. A connector is used for separating the guide wire lumen and the balloon inflating lumen. The balloon catheter shaft carries the guide wire lumen and the balloon inflating lumen separately. Ring markers on the catheter shaft are used so that the start of balloon tapers and the edges of the stent can be visualized by X-ray.

Conveniently, the delivery catheter can be a conventional balloon dilation catheter used for angioplasty procedures. The balloon can be formed of suitable materials such as irradiated polyethylene, polyethylene terephthalate, polyvinylchloride, nylon, and copolymer nylons such as PEBAX™. Other polymers may also be used. In order for the stent to remain in place on the balloon during delivery to the desired site within an artery, the stent is typically crimped onto the balloon. However, the precise design choices in delivery systems are not limiting to the scope of the disclosure.

A vessel blockage can be generally defined as any obstruction of an artery in a human, which obstruction can be caused by either atherosclerosis, a blood clot, plaques, or other biologically originating. A blood clot is commonly referred to as a thrombus, if it formed at the site of the occlusion, or an embolus, if it formed in another area of the cardiovascular system and traveled to the site of the occlusion.

Atherosclerosis is a specific form of arteriosclerosis in which an artery wall thickens as a result of the accumulation of fatty materials such as cholesterol and triglycerides. It is caused by formation of multiple plaques within arteries. An atherosclerotic plaque is divided into three distinct components: (1) the atheroma nearest to the lumen which is a nodular accumulation of soft yellowish material composed of macrophages; (2) an underlying area of cholesterol crystals, and (3) calcifications located at the outer edge of the plaque and nearest the vessel wall, present mainly in older lesions.

Atherosclerotic lesions are separated into two broad categories: stable and unstable lesions. Stable lesions are generally rich in extracellular matrix and smooth muscle cells. Unstable lesions are rich in lipid-loaded macrophages and usually have a weak fibrous cap, which separates the plaque from the blood vessel lumen. The weak fibrous cap is prone to rupture. Upon cap rupture, thrombogenic material, such as collagen, is exposed to the circulating blood and can induce thrombus formation. Upon formation, intraluminal thrombi can occlude vessels outright and cause infarction of tissues or organs dependent on blood supply by the occluded vessel. Alternatively, thrombi may detach and move into the blood circulation and eventually occlude smaller downstream vessel branches causing thromboembolism.

The present invention provides a device and methods for removing blood vessel blockages, including, but not limited to, blood clots, thrombi and emboli. In certain embodiments, the device comprises a wire or wire-like structure, wherein the wire is made of biocompatible material, which can generate heat, can be used to elevate the temperature of a vessel blockage, and enables the device to capture the blockage with minimal force applied to the vessel wall. In further embodiments, the wire is made of biocompatible metals.

The biocompatible metals can include, but are not limited to, titanium, titanium alloy, magnesium, magnesium alloy, tungsten, tungsten alloy, zinc, zinc alloy, aluminum, aluminum alloy, iron, iron alloy, steel, manganese, manganese alloy, calcium, calcium alloy, zirconium, and zirconium alloy.

The method of blockage removal can include navigation to the site of the blockage, integration of the wire into the blockage, temperature elevation of the blockage, and extraction of the blockage material. In one embodiment, the device is navigated to the position of the targeted blockage inside a vessel by a guide wire. In one embodiment, a guide wire is inserted into the vessel using endovascular catheterization techniques. The guide wire can be located in a position adjacent to the targeted blockage. The guide wire can be used to penetrate into the blockage for a desired distance. In a further embodiment, a microcatheter, which carries the wire of the present invention, is advanced through the catheter shaft. The wire of the present invention can be deposited from the microcatheter into the vessel lumen so as to be adjacent to the vessel blockage. The deployed wire can then be advanced to integrate with the blockage. After integrating with the blockage, the wire can elevate the temperature of the blockage for a predetermined period of time. The elevated temperature results in an increase in the consistency of the blockage material and a stable interaction between the wire structure and the blockage material. The wire structure integrated with the blockage is then retrieved to the catheter and removed from the vessel.

The devices of the subject invention can be used along or in combination with an angioplasty balloon or a stent. Ideally, the devices of the subject invention are utilized to remove occlusion material and a stent is emplaced to maintain the lumen of the vessel.

In one embodiment, the wire-like structure of the present invention is a wire loop. In a preferred embodiment, the wire loop is coated with one or more electrically conducting and/or insulating materials.

In certain embodiments, the wire loop is coated with one or more layers of coating to prevent direct contact between the wire or wire-like structure and the vessel wall, which may transfer electrical energy to, or overheat the vessel wall. These layers of coating can be made of one or more biocompatible materials, including, but not limited to, thermal insulated materials, electrical insulated materials or thermal and electrical insulated materials.

In one embodiment, the wire or wire-like structure is made of biocompatible metal coated with a biocompatible material loaded with a self-heating chemical. In another embodiment, the wire is coated with a material loaded with a combination of two or more chemicals that can generate heat when combined.

In one embodiment, the wire body is heated by electricity. In another embodiment, the wire body emits microwaves to heat the surrounding occlusion materials.

In one embodiment, the wire coating comprises a first component comprising a thermal insulated coating and a second component comprising an electrical insulated coating.

In one embodiment, the wire coating comprises a first component comprising a thermal insulated coating and a second component comprising a self-heating chemical or a combination of chemicals that generate heat when being combined.

In one embodiment, the wire loop coating comprises a first component comprising a thermal insulated coating and a second component comprising a microwave-heatable material.

In one embodiment, the wire loop coating comprises a first component comprising an electrical insulated coating, a second component comprising a thermal insulating coating, and a third component comprising a self-heating chemical or a combination of chemicals that generate heat when being combined.

In one embodiment, the wire loop coating comprises a first component comprising an electrical insulated coating, a second component comprising a thermal insulating coating, and a third component comprising a microwave-heatable material.

In one embodiment, the wire loop made of biocompatible and electrically heatable metal is heated by applying incremental portions of electrical energy to the wire loop.

In one embodiment, the wire loop is made of biocompatible, microwave-emitting material and a microwave-heatable coating material is heated by applying incremental portions of microwave energy to the microwave-heatable coating material.

Following is an example that illustrates a procedure for practicing the invention. This example should not be construed as limiting.

### EXAMPLE 1

### Removal of Vessel Blockage

An ex vivo model of blood clot removal was designed using a cannula, coagulated blood inside the cannula, and a wire structure device. The wire structure of the present device was integrated with the blood clot inside the cannula. Electricity was applied to the wire structure for heating purposes. The solidified blood clot was retrieved through the cannula. Subsequently, solution injected into the cannula could flow freely through the recanalized cannula.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A device for removal of a target blockage such as a thrombus, blood clot, or embolus in a vessel, the device comprising a wire or a wire-like structure made of biocompatible material, wherein the device transmits electrical or thermal energy to a target blockage in a vessel when in use.

2. A device according to claim 1, further comprising a biocompatible coating that is made of electrical insulator.

3. A device according to claim 1, further comprising a biocompatible coating that is loaded with self-heating chemical or combinations of chemicals that can generate heat when being combined.

4. The device of claim 1 any of claim 1, further comprising a partially coated biocompatible coating in the outermost layer that comprises an electrical or thermal insulator.

5. The device of claim 1, wherein the biocompatible material can be heated by electricity or microwave.

6. The device of claim 5 wherein the biocompatible coating in the outermost layer of the device inhibits the wire or wire structure from contacting vessel wall.

7. The device of claim 4 wherein the biocompatible coating is loaded with self-adhering chemical or combinations of chemicals that generate adhesion when being combined, thereby enhancing interfacial adhesion between the device and a target blockage.

8. A method of removal of an occlusion existing in a vessel, comprising navigating the device of claim 1 inside a vessel to reach a target occlusion in the vessel; integrating the device with the target occlusion; and removing the device and the occlusion from the vessel.

9. A device for removing vessel occlusions comprising: a wire having a terminal end, wherein the terminal end conveys an electrical current or generates heat and wherein there is at least one of a thermally insulating coating or an electrically insulating coating covering at least a portion of the terminal end.

10. A device according to claim 2, further comprising a biocompatible coating that is loaded with self-heating chemical or combinations of chemicals that can generate heat when being combined.

11. The device of claim 2, further comprising a partially coated biocompatible coating in the outermost layer that comprises an electrical or thermal insulator.

12. The device of claim 3, further comprising a partially coated biocompatible coating in the outermost layer that comprises an electrical or thermal insulator.

13. The device of claim 2, wherein the biocompatible material can be heated by electricity or microwave.

14. The device of claim 3, wherein the biocompatible material can be heated by electricity or microwave.

15. The device of claim 4, wherein the biocompatible material can be heated by electricity or microwave.

16. A method of removal of an occlusion existing in a vessel, comprising navigating the device of claim 2 inside a vessel to reach a target occlusion in the vessel; integrating the device with the target occlusion; and removing the device and the occlusion from the vessel.

17. A method of removal of an occlusion existing in a vessel, comprising navigating the device of claim 3 inside a vessel to reach a target occlusion in the vessel; integrating the device with the target occlusion; and removing the device and the occlusion from the vessel.

18. A method of removal of an occlusion existing in a vessel, comprising navigating the device of claim 4 inside a vessel to reach a target occlusion in the vessel; integrating the device with the target occlusion; and removing the device and the occlusion from the vessel.

19. A method of removal of an occlusion existing in a vessel, comprising navigating the device of claim 5 inside a vessel to reach a target occlusion in the vessel; integrating the device with the target occlusion; and removing the device and the occlusion from the vessel.

20. A method of removal of an occlusion existing in a vessel, comprising navigating the device of claim 6 inside a vessel to reach a target occlusion in the vessel; integrating the device with the target occlusion; and removing the device and the occlusion from the vessel.

21. A method of removal of an occlusion existing in a vessel, comprising navigating the device of claim 7 inside a vessel to reach a target occlusion in the vessel; integrating the device with the target occlusion; and removing the device and the occlusion from the vessel.
